(19) United States
(12) Patent Application Publication
(10) Pub. No.: US 2026/0043798 A1
(43) Pub. Date: Feb. 12, 2026

(54) LIQUID TESTING CARTRIDGES, ASSOCIATED APPARATUS AND METHODS

(71) Applicant: FREDsense Technologies Corp., Calgary (CA)

(72) Inventors: Robert Mayall, Calgary (CA); Margaret Renaud-Young, Calgary (CA); Cheng Chen, Calgary (CA); Mhairi McDonald, Calgary (CA); Sergio Sanchez Garcia, Calgary (CA); Emily Candice Hicks, Calgary (CA)

(73) Assignee: FREDsense Technologies Corp., Calgary (CA)

(21) Appl. No.: 19/360,243

(22) Filed: Oct. 16, 2025

Related U.S. Application Data
(63) Continuation of application No. PCT/CA2024/050496, filed on Apr. 17, 2024.
(60) Provisional application No. 63/460,188, filed on Apr. 18, 2023.
(60) Provisional application No. 63/610,171, filed on Dec. 14, 2023.

(51) Int. Cl. G01N 33/543; B01L 3/00; G01N 27/416

(52) U.S. Cl. CPC G01N 33/54386; B01L 3/502761; G01N 27/4163; G01N 33/5438; B01L 2200/04; B01L 2200/0647; B01L 2300/0663; B01L 2300/087

(57) ABSTRACT

A cartridge, kit and method of determining the presence of a target material in a fluid sample are disclosed. The cartridge comprises multiple reservoirs, each reservoir comprising: a processing compartment in fluid communication with an inlet; and a sensor compartment with a sensor for generating a response to the presence of a detectable material. The detectable material is produced by a processing material from a detectable material precursor when the target material is present. This allows a wide range of assays, including bacterial assays and ELISAs to be used in the field. The cartridge may be used in conjunction with a base to calibrate the response of the sensor to known quantities of target material.

### CROSS-REFERENCE TO RELATED APPLICATIONS

This application, filed under 35 U.S.C § 111(a), is a continuation of PCT Application No. PCT/CA2024/050496, filed on Apr. 17, 2024, which claims the benefit of U.S. Provisional Patent Application No. 63/460,188, filed on Apr. 18, 2023 and to U.S. Provisional Patent Application No. 63/610,171, filed on Dec. 14, 2023. The above-referenced patent applications are herein incorporated by reference in their entireties.

### TECHNICAL FIELD

The invention relates to measuring the presence and/or concentration of a target material in a fluid sample. In particular, the invention relates to detecting target materials in the field using a robust field kit.

### BACKGROUND

The ELISA (Enzyme-Linked ImmunoSorbent Assay) is a widely used assay platform for the specific recognition of unique molecular signatures. However, they are multi-step laboratory processes requiring the use of multi-well plates, micropipettes, multiple reagents, several solution changes, and a light-based instrument to measure the outcome. This complexity limits the scalability and makes it such that they are performed in well-equipped research and service labs and are not easily translatable to onsite remote testing. There are multiple instances in which the portability of this assay would be an asset to a user.

Scalability has been addressed by the availability of 8- or 12-well strips of ELISA plates. Another common approach is to use microfluidic systems.

Another method of detecting materials is to use bacterial biosensors. A bacterial biosensor uses bacteria to produce a detectable material in response to the presence of an analyte. A sensor can then be used to detect the detectable material to generate a measurable signal.

### RELEVANT CO-OWNED PATENTS AND PATENT APPLICATIONS

U.S. Patent Application Publication US 2022/120705 published on Apr. 21, 2022, owned by FREDsense Technologies Corp., and hereby incorporated by reference in its entirety, discloses cartridges for the voltammetric detection of fluid parameters in a fluid sample are provided. The cartridges contain a sample reservoir containing two compartments fluidically separated by a barrier. Each compartment contains a chemical compound to facilitate voltammetric detection of a fluid parameter. A fluid collection device containing a fluid sample can be received by the sample reservoir, and the barrier can be penetrated by the fluid collection device, to thereby cause contact between the fluid sample and both chemical compounds. Upon introduction of the fluid sample in the sample reservoir a fluid parameter can be voltammetrically detected. Related assemblies including the cartridges, as well as methods for operating the cartridges are also described.

U.S. Pat. Nos. 9,689,046 and 10,415,102 granted on Jan. 27, 2017, Sep. 17, 2019 respectively, owned by FREDsense Technologies Corp., and hereby incorporated by reference in its entirety, disclose methods that may be used for the electrochemical detection of multiple parameters, including chemical compounds. Further provided are cells that may be used in the electrochemical detection of multiple parameters, including chemical compounds, as well as a kit for the electrochemical detection of multiple parameters, including chemical compounds.

U.S. Patent Application No. 63/455,650 filed on Mar. 30, 2023, and PCT Patent Application number PCT/CA2024/050395 filed on Mar. 28, 2024, both owned by FREDsense Technologies Corp., and hereby incorporated by reference in their entirety, disclose antibodies and antibody fragments that bind to polyfluoroalkyl and perfluoroalkyl substances (PFAS) for use in biosensors for detection of PFAS compounds in environmental samples. Detectors incorporating the biosensors and kits for use with the detectors are also described.

U.S. Patent Application No. 63/610,171 filed on Dec. 14, 2023, owned by FREDsense Technologies Corp., and hereby incorporated by reference in its entirety, discloses a method for detecting and quantifying a PFAS compound or a mixture of PFAS compounds in a sample. The method includes the steps of providing a complex of a cyclodextrin and an indicator molecule, contacting the complex with the sample and measuring a parameter of the indicator molecule.

### SUMMARY

In accordance with the present disclosure, there is provided a kit comprising:
a processing material;
a detectable material precursor; and
a sample analysis cartridge for the detection of a target material in a fluid sample, the sample analysis cartridge comprising:
multiple reservoirs, each reservoir comprising:
   an inlet for receiving a fluid sample;
   a processing compartment in fluid communication with the inlet; and
   a sensor compartment being in fluid communication with the processing compartment and being accessible by a sensor, the sensor being configured to provide an output in response to the presence of a detectable material, the detectable material being producible by the processing material from the detectable material precursor,
wherein the kit is configured such that the quantity of detectable material produced by the processing material is correlated to the quantity of the target material in the fluid sample.

The sensor may comprise a voltammetric sensor.

The sensor may comprise an optical sensor.

The sensor compartment may be accessible by a sensor by containing and/or comprising the sensor. The sensor compartment may be accessible by an optical sensor by comprising a being transparent, or by having a transparent window. The sensor compartment may be accessible to a voltammetric sensor by containing or comprising electrodes with connectors which connect to the exterior of the sample analysis cartridge. The electrode connectors may allow the electrodes to be electrically connected to other sensor circuitry.

In the context of this disclosure, accessible by a sensor may mean that the sensor can interact with the contents of the sensor compartment to detect an output in response to the presence of a detectable material. An optical sensor may detect the intensity of one or more frequencies of light emitted by the contents of the sensor compartment. A voltammetric sensor may detect a voltammetric response of the contents of the sensor compartment.

In the context of this disclosure, accessible by a sensor may mean that the sensor can probe the contents of the sensor compartment. An optical sensor may probe the contents of the sensor compartment by illuminating the contents of the sensor compartment. A voltammetric sensor may probe the contents of the sensor compartment by applying a voltage across and/or a current between electrodes in contact with the contents of the cartridge.

The cartridge may comprise voltammetric sensor electrodes.

A binding material may be immobilised on a surface of the reservoir, the binding material being configured to selectively bind to the target material. A binding material may be immobilised on surface contained by the reservoir (e.g., on beads or on a surface inserted into the reservoir).

The binding material may be an antibody or antibody fragment suitable for use in an ELISA.

The binding material may comprise at least one of: an IgG (Immunoglobulin G) antibody; and a VHH antibody.

The processing material may be a cyclodextrin, and the detectable material precursor may be a guest molecule bound to the cyclodextrin in a complex, the guest molecule being displaceable by the target material to form an unbound guest molecule detectable material.

The processing material may comprise bacteria. The bacteria may be genetically modified bacteria configured to be responsive to the target material. The processing material may be a whole-cell biosensor, which is typically a genetically modified bacteria that couples induction of the target-material-responsive promoter to the expression of a reporter gene for a quantifiable output.

The biosensor may be based on the ars promoters of Escherichia coli (K-12 genome and R773 plasmid) Staphylococcus plasmid pI258 and Bacillus subtilis genome with reporters such as β-galactosidases (e.g., LacZ), fluorescent proteins (e.g., GFP), or luciferases (e.g., Lux), or other enzymes.

The sensitivity of the kit may be dependent on the target material. The kit may have a target material specificity and sensitivity under 10 ppm. The kit may have a target material specificity and sensitivity under 10 ppb. The kit may have a target material specificity and sensitivity under 10 ppt. In the present disclosure, unless stated otherwise, ratios, such as ppm, ppb and ppt values, are given in terms of mass by volume (e.g., mg/L for ppm).

Each reservoir may be connected to adjacent reservoirs by a single layer impermeable wall. This helps thermal conductivity between adjacent reservoirs so that all the reservoirs have the same temperature.

The cartridge may comprise and/or contain the processing material. E.g., the processing material may be provided in the cartridge. The processing material may be provided in a dried and/or cured form. The user may rehydrate the dried or cured processing material by adding water (e.g., as part of the sample).

The cartridge may comprise and/or contain the detectable material precursor. E.g., detectable material precursor may be provided in the cartridge. The detectable material precursor may be provided in a dried and/or cured form.

The cartridge may comprise and/or contain a buffer solution, or a dried buffer material configured to form a buffer solution when mixed with water.

The target material may comprise one or more of: arsenite; arsenic; arsenide; arsenate; selenium; iron; Fe(II); Fe(III); manganese; BTEX; PFAS; saxitoxins; microcystins; gonyautoxins; dioxanes; and chromate.

The sensor compartment may comprise a planar inner surface on which the sensor (e.g., the electrodes) is mounted, and a parallel opposing surface.

The volume of the processing compartment may be at least 5 times the volume of sensor compartment.

The volume of each reservoir may be less than 3 ml.

The volume of the sensor compartment may be between 100-300 μL.

The processing compartment may be positioned between the sensor compartment and the inlet.

The sample analysis cartridge may be rotationally asymmetric.

The sample analysis cartridge may have mirror symmetry with at least one pair of reservoirs arranged on either side of a mirror plane.

The cartridge may comprise between 4 and 8 reservoirs. The cartridge may comprise 6 reservoirs.

The cartridge may comprise at least one test reservoir, and at least one calibration reservoir,
wherein the kit comprises a target material remover configured to remove the target material from a sample to form a calibration fluid for injection into the calibration reservoir,
and wherein a known amount of target material is present in the calibration reservoir, and wherein the test reservoir is configured to receive an unaltered sample fluid.

Each reservoir may comprise a visual code associated with whether the reservoir is a test reservoir or a calibration reservoir.

The reservoirs may have a common central barrier, the barrier comprising the sensor for each of the reservoirs.

The sample analysis cartridge may have a tapered external shape wherein the width of the cartridge around the sensor compartment is narrower than the width of the sample analysis cartridge around the processing compartment.

Each reservoir may comprise a transparent region. The entire reservoir may be formed from a transparent material. The reservoir may comprise a transparent window. The top of the reservoir may be transparent. A transparent window may allow for optical measurement by an optical sensor. The optical sensor may interact with the cartridge via an optical fibre.

The sample analysis cartridge may comprise snap-fit components connected together.

The sample analysis cartridge may comprise injection-molded components connected together.

According to a further embodiment, there is provided a method of detecting a target material in a fluid sample using a processing material, a detectable material precursor, and a sample cartridge, the sample analysis cartridge comprising:
multiple reservoirs, each reservoir comprising:
an inlet for receiving a fluid sample;
a processing compartment in fluid communication with the inlet; and
a sensor compartment being in fluid communication with the processing compartment and being accessible by a sensor, the sensor being configured to provide an output in response to the presence of a detectable material, the detectable material being producible by the processing material from the detectable material precursor,
the method comprising:
injecting the fluid sample into the reservoir;
enabling production, by the processing material, of the detectable material from the detectable material precursor within the reservoir based on the presence of a target material; and
detecting the response of the sensor to the detectable material.

The method may comprise:
injecting the fluid sample into the reservoir, the reservoir comprising a binding material configured to selectively bind the target material within the injected sample to the surface within the cartridge;
flushing the cartridge;
adding the processing material and the detectable material precursor;
allowing the detectable material precursor fluid to interact with the intermediate material to produce the detectable material;
detecting the response of the sensor to the detectable material.

The method may comprise:
injecting the fluid sample into the reservoir;
adding the processing material to the fluid sample;
allowing the processing material to interact with the sample to produce the detectable material;
detecting the response of the sensor to the detectable material.

The method may comprise:
simultaneously determining the quantity of target material within multiple reservoirs within a cartridge, wherein at least two of the determinations are calibration determinations, and at least one determination is a test determination,
wherein each calibration provides an association between a known quantity of target material and a sensor response; and
wherein the sensor response of the at least one test determination is interpolated between the sensor responses of the at least two calibration determinations to provide a concentration of the target material.

The method may comprise removing the target material from a sample fluid to form a calibration fluid, wherein the calibration fluid is used in the calibration determination, and the unaltered sample fluid is used in the test determination.

Removing the target material from the sample fluid to form the calibration fluid may comprise one or more of: filtration, reacting the target material, changing the oxidation state of the target material, changing the physical state of the target material, immobilising the target material, binding the target material and chelating the target material.

According to a further aspect, there is provided a sample analysis cartridge for the detection of a target material in a fluid sample, the cartridge comprising:
multiple reservoirs, each reservoir comprising:
an inlet for receiving a fluid sample;
a processing compartment in fluid communication with the inlet; and
a sensor compartment being in fluid communication with the processing compartment and being accessible by a sensor, the sensor being configured to provide an output in response to the presence of a detectable material, the detectable material being producible by a processing material from a detectable material precursor based on the presence of a target material.

The reservoir may contain one or more of: the processing material; the detectable material precursor; and a binding material configured to selectively bind the target material to a surface within the cartridge.

According to a further aspect, there is provided a sample analysis assembly comprising: the sample analysis cartridge as described herein, wherein the sensor comprises a voltammetric sensor, and a base (or base unit), the base comprising electronic connectors for connecting to the voltammetric sensor.

The base may comprise the sensor.

The base may have a recess with a complementary shape to engage with the external surface of the sample analysis cartridge.

The sample analysis cartridge may have mirror symmetry and be rotationally asymmetric.

The cartridge may comprise one or more indents. Indents may be positioned between two adjacent reservoirs, the indents being positioned away from the centre along a reservoir axis.

The base may comprise a battery for powering electronics and controlling the voltammetric sensor.

The electronics may comprise a potentiostat. A potentiostat may comprise the electronic hardware required to control an electrochemical cell. A potentiostat may be used to control a two-electrode system. A potentiostat may be used to control a three-electrode system.

The base may comprise a heater, the heater comprising complementary surfaces to the external surfaces of the sample analysis cartridge.

The base may be configured to identify which reservoirs are being used to perform a calibration determination, and which reservoirs are being sued to perform a test determination, and wherein the base is configured to calculate a concentration of the target material based on the response of the sensor in the test determinations, and on the response of the sensor in each of the calibration determinations.

Each reservoir may comprise an inlet and an outlet. The inlet may comprise a one-way valve.

The present technology may enable ELISAs to be performed in the field without the need for significant training or for the investment in laboratory grade equipment. The present technology may be used to detect PFAS molecules, environmental contaminants (e.g., metals such as arsenic, antimony, cadmium, chromium, copper, lead, selenium etc.), pesticides, viruses (e.g., COVID-19), toxins, bacteria, etc.

The apparatus may used to perform an ELISA. An ELISA may be one or more of: a direct ELISA; an indirect ELISA; a sandwich ELISA; and a competitive ELISA.

In an ELISA, the processing material may comprise an antibody and attached enzyme. In a direct ELISA, the processing material may be a primary antibody conjugate (e.g., with an attached enzyme). In an indirect ELISA, the processing material may have two components: a primary antibody conjugate configured to bind to the target material, and a secondary antibody conjugate (e.g., with enzyme) configured to bind to the primary antibody conjugate. A sandwich ELISA may have processing materials described in relation to the direct and indirect ELISAs. A sandwich ELISA may also have a binding material which may be a capture antibody which is specific to the target material and is immobilised within the reservoir.

In a competitive ELISA, the processing material may be a labelled reference processing material comprising the target material bound to an enzyme (e.g., via an antibody). A competitive ELISA may also use a binding material (e.g., a capture antibody). The binding material may bind to the target material in the sample, and to the target material in the labelled reference processing material. In this way, there is a competition between the target material in the labelled reference and the target material in the sample to bind to the binding material.

Another method of competitive ELISA is to have the analyte/target material (sometimes in a modified but still bindable form, e.g., conjugated to something that sticks well to the surface) immobilized in the reservoir. The processing material (e.g., comprising a target-specific antibody and enzyme) is then mixed with the sample and exposed to the surface with the binding material comprising the target material, so the antibody has the choice to bind to either the surface-confined target material or the free (unbound) target material in the sample. This creates an equilibrium where, in a concentration-dependent manner, how much free target molecule is in a sample can be quantified by observing how much becomes surface-confined (after washing away any unbound antibodies).

In direct, indirect, sandwich and competitive ELISAs, the detectable material precursor may be a substrate, such as p-aminophenol phosphate. In direct, indirect, sandwich and competitive ELISAs, the detectable material may be an electroactive material such as 4-aminophenol.

In a bacterial assay, the processing material may be the bacteria itself. In a bacterial assay, the detectable material precursor may be a substrate, such as PAPG. The detectable material may be an electroactive material such as 4-aminophenol. As the same detectable material may be used in both ELISA and bacterial assays, the same sensor may be used in a wide range of assays.

In a bacterial assay, the bacteria interact with the target material within the sample and produce a specific protein (e.g., in a preferred embodiment this is a beta-galactosidase protein) that reacts with a detectable material precursor substrate (4-aminophenyl-beta-D-galactopyranoside, PAPG) to produce galactose and 4-aminophenol. There are multiple methods of adding a detectable material precursor, such as PAPG, into the cartridge reservoir. One is to inject the detectable material precursor as a liquid. A second is to deposit the detectable material precursor onto a surface and have it in a cured or dry format. The surface may comprise a porous matrix (e.g., paper that is added to the cartridge), or an impermeable surface (e.g., a plastic surface inside the reservoir).

The kit may be used to perform a displacement assay. In a displacement assay, the processing material and detectable material precursor may comprise a complex (e.g., a host-indicator complex) of a host molecule (e.g., the processing material) and a guest molecule (e.g., detectable material precursor). The host molecule may be a cyclodextrin. The guest molecule, when bound to the host, may be a detectable material precursor. The guest molecule, when unbound from the host molecule may be a detectable material. The bound guest molecule may respond differently to the sensor than the unbound guest molecule.

When the complex is mixed with a sample containing the target compound, the target compound may displace the bound guest molecule detectable material precursor to generate an unbound guest molecule detectable material, thereby changing the response of the mixture. In this way, the unbound guest molecule may act as the detectable material, which is detectable by measuring a parameter associated with the different response. It will be appreciated that the unbound guest molecule may provide a stronger or a weaker response to the sensor than the bound guest molecule. In displacing the bound guest molecule, the target molecule may bind with the host molecule to form a host-target complex.

The guest molecule may be a fluorescent molecule which exhibits differences in fluorescence when it is bound to the host molecule than when it is unbound from the host molecule. The guest molecule may be an electroactive molecule which exhibits differences in voltammetric or other electrochemical response when it is bound to the host molecule than when it is unbound from the host molecule.

The target molecule for a displacement assay may be a PFAS compound.

The cyclodextrin may be α-cyclodextrin, β-cyclodextrin, or γ-cyclodextrin and may include one or more non-hydrogen substituents.

The cyclodextrin may be selected from the group consisting of: 2-hydroxypropyl-β-cyclodextrin, triacetyl-β-cyclodextrin and 6-amino-β-cyclodextrin.

In some embodiments, the cyclodextrin is β-cyclodextrin polymer (p-β-cyclodextrin). The β-cyclodextrin polymer comprises a linker. The linker may be a cyclic ether. The cyclic ether may be a substituted or unsubstituted oxacyclopropane, oxacyclobutane, oxacyclopentane, oxacyclohexane or furan.

In some embodiments, the β-cyclodextrin polymer comprises at least one non-hydrogen substituent on at least one glucopyranoside unit. The non-hydrogen substituent may be any one of or a combination of hydroxypropyl, acetyl, azido, hydroxyamino and amino.

In some embodiments, the indicator molecule is a dye molecule. The dye molecule may be a colorimetric dye molecule or a fluorescent dye molecule. The fluorescent dye molecule may be 9-(diethylamino)-5H-benzo[a]phenoxazin-5-one) (Nile red), SYPRO™ Orange, or 8-anilinonaphthalene-1-sulfonic acid (ANS).

In some embodiments, the fluorescent dye is provided at a concentration between about 0.0001 mM and about 0.015 mM.

In some embodiments, the cyclodextrin is p-β-CD, which is provided at a concentration between about 0.1 mM and about 0.5 mM.

In some embodiments, the indicator molecule is an electroactive molecule. The electroactive molecule may be a metallocene or an electroactive derivative thereof, or a para-substituted phenol or an electroactive derivative thereof. The metallocene or the electroactive derivative thereof, may be ferrocene, ferrocenemethanol, ferrocenedimethanol, ferrocenecarboxylic acid, ferrocenecarboxaldehylde, or aminoferrocene. The indicator may be para-aminophenol (PAP).

In some embodiments, the electroactive molecule may be provided at a concentration of about 0.1 mM to about 0.5 mM.

Typical cyclodextrins contain a number of glucose monomers ranging from six to eight units in a ring, creating a cone shape. Common examples include α-(alpha)-cyclodextrin, which has 6 glucose subunits, β-(beta)-cyclodextrin, which has 7 glucose subunits, and γ-(gamma)-cyclodextrin, which has 8 glucose subunits. The cyclodextrins have toroidal shapes, with the larger and the smaller openings of the toroid exposing to the solvent secondary and primary hydroxyl groups respectively. Because of this arrangement, the interior of the toroid is not completely hydrophobic, but considerably less hydrophilic than the aqueous environment and thus able to host other hydrophobic molecules, thereby forming what is known as an inclusion complex or a host-guest complex. In contrast, the exterior is sufficiently hydrophilic to provide the cyclodextrins with solubility in water and other polar solvents.

Without being bound by any particular theory, the inventors postulate that a cyclodextrin with high affinity for PFAS compounds may be provided when the cyclodextrin is provided with one or more functional groups which can interact with the head group of a given PFAS compound (for example —COOH or —SO₃ if PFOA or PFOS, respectively) or with the backbone (i.e., —CF₂— units). In one embodiment, there is provided a cyclodextrin modified with a long 13 CF₂— tail on one end of the toroid of the cyclodextrin to allow the cyclodextrin to interact with long chain PFAS (for example, greater than ten carbons in length, such as perfluorododecanoic acid, which is 12 carbons long). In another embodiment, strong positive charges provided by functional groups such as —NH₂, —NH₃⁺, or quaternary amines such as —NR₃⁺), to promote interactions with the negatively charged acid or sulfonyl groups. In other embodiments, where the cyclodextrin is in polymeric form, the monomer units are connected by linkers which include functional groups that can increase affinity for PFAS compounds. Examples of such linker modifications are described in Wang et al., ACS Central Science, 2022, 8, 663, which is incorporated herein by reference in its entirety.

PFAS compounds may be classified into subsets according to their chemical properties. There is currently a strong need to detect compounds such as PFOA and PFOS, which are negatively charged at neutral pH. It is expected that detection of positively charged PFAS compounds will become important as well, as this subset of PFAS is newer and less well regulated. A cyclodextrin with anionic functional groups to provide a negatively charged cyclodextrin could be used to improve affinity for positively charged PFAS compounds. For detection of zwitterionic PFAS compounds, the inventors have conceived of a solution by designing a cyclodextrin with suitable spacing of carboxylic acid and amino groups to increase the affinity of the modified cyclodextrin for a given zwitterionic PFAS compound, to lower the detection limit of an indicator displacement assay. In another embodiment, a pair of cyclodextrins could be used to create a sensor for cationic and anionic PFAS by having respective carboxylic and amino substituents on their outer surfaces such that the amino-modified cyclodextrin is positively charged at pH 7 while the carboxyl-modified cyclodextrin is negatively charged at pH 7. This combination of cyclodextrins will be useful for detecting zwitterionic PFAS compounds in the indicator displacement assay. While some of these modifications are known in the art, they are described in terms of absorption and remediation and have not been explored in terms of development of an assay to use as a sensor for quantification of PFAS compounds. It is to be understood that such modifications may be applied to cyclodextrin monomers and polymers.

The present technology may use indicator molecules to provide a measurable parameter which indicates binding of a PFAS compound. In this context, “binding” refers to entry of another molecule into the cavity of a cyclodextrin in a host-guest interaction, which is presumed to be dominated by hydrophobic interactions and other non-covalent interactions. The parameter provided by the indicator molecule may be any measurable physical parameter. Examples of such parameters of indicator molecules include, but are not limited to, color change (colorimetric analysis), fluorescence, and change in oxidation state (redox chemistry). Indicator molecules for colorimetric analysis and fluorescence detection include dye molecules (also known as chromophores or fluorophores), which have conjugated multi-ring structures. Some examples of fluorescence indicator dyes include, but are not limited to: SYPRO™ Orange (Thermo Fisher (S6650), fluorescein, rhodamine, cyanine, BODIPY-FL, 7-nitrobenz-2-oxa-1, 3-diazole-4-yl, naphthalimide (lucifer yellow), and acridine orange. Indicator molecules for redox detection are also known as electroactive analytes. Examples of such electroactive analytes are described in U.S. Pat. Nos. 9,689,046 and 10,415,102. Additional examples include metallocenes containing metals such as iron, cobalt, chromium, nickel ruthenium and vanadium. Ferrocene and derivatives such as ferrocenemethanol, ferrocenedimethanol, ferrocenecarboxaldehyde, ferrocenecarboxylic acid, and aminoferrocene are expected to be useful as electroactive compounds in the indicator displacement assays described herein. Such compounds are capable of being reduced and/or oxidized when a voltage is applied to an electrical cell and provide a measurable electrical current in the electrical cell. Other examples of indicator molecules may be bifunctional by providing a chromophore while being electroactive. Some embodiments may include a mixture of indicators to provide a visual quality control metric to a user while a more quantifiable result is produced electrochemically using an electroactive indicator molecule.

An indicator displacement assay is based on a supramolecular assembly of an indicator molecule that is reversibly bound to a host molecule. In the presence of an analyte, the indicator molecule is displaced from the host, resulting in a measurable change in a physical parameter. The target analyte must also have a higher affinity at a particular concentration. The term “affinity” refers to the extent that the target analyte is bound relative to the indicator molecule, and thus the affinity is related to the association constant (sometimes referred to as the “formation constant”) and concentration of the host, guest, and indicator molecule, to achieve an effective displacement of the indicator and provide a measurable change of a parameter (Sedgewick et al. Chem. Soc. Rev., 2021, 50, 9). The change of the parameter will indicate the amount(s) of the target analyte(s) in the sample being assayed.

The kit may comprise pretreatment materials. The kit may comprise calibration pretreatment materials for preparing the sample for calibration determinations, the kit may comprise test pretreatment materials for preparing the sample for test determinations.

The sample analysis assembly may provide a detectable output interface to provide a readout to the user (e.g., having a screen for graphically displaying the result). The sample analysis assembly may provide an electrochemical readout.

The fluid sample may be an aqueous sample. The fluid sample may comprise at least 90% water by mass.

The cartridge may have an integrated screen-printed electrode sensors to enable measurement inside of the system. The sensor may be one or more of: an electrochemical sensor, a colourimetric sensor; a luminescent sensor; a fluorescent sensor; and an optical sensor. A colourimetric detectable material may allow the user to determine that the correct procedure has been carried out and/or that the materials have not expired through visual inspection.

Each reservoir may comprise a transparent window. A transparent window to view the contents of a reservoir may which help the user understand which buffers/solutions to add where and/or it may be an easy way to see if something was added or not.

A media dispenser may be a pipette or a syringe. A syringe may comprise a blunted plastic cannula.

A lateral dimension between the outer walls of the cartridge around the sensor compartment may be 60% or less than the lateral dimension of the between the outer walls of the cartridge around the processing compartment. A lateral dimension between the outer walls of the cartridge around the sensor compartment may be 50% or less than the lateral dimension of the between the outer walls of the cartridge around the processing compartment. This may allow for more efficient heating of the cartridge during any incubation stages. During incubation stages, the inlet of the reservoirs may be closed with a lid.

The lateral outer walls of the reservoir may be less than 2 mm thick. This may allow for more efficient heating of the cartridge during any incubation stages.

The walls of the cartridge may be formed from a polymer such as polycarbonate or polystyrene. The walls of the cartridge may be formed from plastic.

In the context of this disclosure, directional words like top, bottom, upper, lower are defined with respect to the cartridge when in use, for example, on a horizontal surface. Generally, the inlets in a cartridge are positioned towards the top of the cartridge.

The cartridge design and material may be compatible with performing an ELISA in the presence of an electrode and could be deployed in a field-compatible detector.

The electrical signal may be detected in accordance herewith using any methodology involving the application of a voltage to the assay medium. The voltage may be applied potentiostatically (i.e., at one voltage), in cyclic voltammetrical fashion (i.e., across a range of defined voltages), or in an alternating current format (i.e., in sinusoidal manner at either a fixed voltage or across a range of defined voltages). This further includes any voltamperometric methodology, including, without limitation, pulse voltammetry, linear sweep voltammetry, chromoamerometry, staircase voltammetry, alternating current voltammetry, impedance spectroscopy, and cyclical voltammetry, and variations or adaptations thereof such as differential pulse voltammetry, or wave-based voltammetry with chronoamperometric steps included in the sweeps.

The processing material may be bacteria. Bacteria may interact with the sample and produce a specific protein (our most preferred embodiment is a beta-galactosidase protein) that reacts with a substrate (4-aminophenyl-beta-D-galactopyranoside, PAPG) to produce galactose and 4-aminophenol. We then detect the produced 4-aminophenol (the detectable material) with electrodes. We have two methods of adding PAPG into the cartridge. The first is to inject it as a liquid, whereas the second is to deposit the PAPG onto a surface and have it in a cured format. This is often done on a porous matrix (e.g., paper) that we add to the cartridge, but can be done on a plastic surface too.

Removing the target material from a sample fluid to form a calibration fluid may comprise physically removing the target material (e.g., by filtering). Removing the target material from a sample fluid to form a calibration fluid may comprise changing or altering the target material such that it no longer influences the processing material. For example, in an arsenite sensor system MnO₂ particles may be used to oxidize arsenite into arsenate so that the arsenite-sensitive bacteria (or processing material) do not detect it. In this case, the MnO₂ particles are large and may be filtered out, but the arsenate (e.g., the altered arsenite) is present in the cartridge. The target material remover may comprise a chemical for altering the chemical state of the target material (e.g., an oxidising or reducing agent). The target material remover may comprise a chemical for binding to the target material (e.g., chelating agent) such that they can be physically separated from the sample and/or not detected by the processing material. The target material remover may comprise a physical component for removing the target material from the sample (e.g., a filter). Removing the target material from a sample fluid may be part of a calibration pretreatment.

The processing and sensor compartments may be different regions of the reservoir.

The heating block may be formed from metal. The heating block may be formed from plastic. The heating block may be formed from a material with a heat conductivity of at least 10 W/mK. The heating block may be formed from a material with a heat conductivity of at least 50 W/mK. The heating block may be formed from a material with a heat conductivity of at least 100 W/mK. The heating block may be formed from a material with a heat conductivity of at least 200 W/mK. The heating block may be formed from a material with a heat conductivity of less than 1,000 W/mK. The heat conductivity may be determined at standard temperature and pressure (e.g., 20° C. and an absolute pressure of 101.325 kPa).

In accordance with the present disclosure, there is provided a use of a cartridge as disclosed herein in an ELISA.

In accordance with the present disclosure, there is provided a use of a cartridge as disclosed herein in a bacterial assay.

In accordance with the present disclosure, there is provided a use of a cartridge as disclosed herein in a displacement assay.

### Antibodies

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins. The antibodies described herein are intended to bind to PFAS compounds.

“Specific binding” is to be understood as via one skilled in the art, whereby the skilled person is clearly aware of various experimental procedures that can be used to test binding and binding specificity. Some cross-reaction or background binding may be inevitable in many protein-protein interactions; this is not to detract from the “specificity” of the binding between antibody and epitope. The term “directed against” is also applicable when considering the term “specificity” in understanding the interaction between antibody and epitope.

Embodiments of antibodies include, but are not limited to polyclonal, monoclonal, bispecific, human, humanized or chimeric antibodies, single variable fragments (ssFv), single domain antibodies (such as VHH fragments from nanobodies), single chain fragments (scFv), Fab fragments, F(ab′)2 fragments, fragments produced by a Fab expression library, anti-idiotypic antibodies and epitope-binding fragments or combinations thereof of any of the above, provided that they retain the original binding properties. Also, mini-antibodies and multivalent antibodies such as diabodies, triabodies, tetravalent antibodies and peptabodies can be generated using the antibodies described herein. Examples of such multivalent antibodies are expected to have enhanced binding affinity for PFAS compounds. The immunoglobulin molecules can be of any class (i.e., IgG, IgE, IgM, IgD and IgA) or subclass of immunoglobulin molecules. Thus, the term antibody, as used herein, also includes antibodies and antibody fragments either produced by the modification of whole antibodies or synthesized de novo using recombinant DNA methodologies.

The present invention further relates to the use of the antibodies, or fragments thereof, as described herein, for example the variable regions, in recognition molecules or affinity reagents that are suitable for selective binding to a target. The antibody or fragment thereof according to the invention may be chemically modified by covalent attachment of chemical or biochemical affinity moieties to the antibody using conventional conjugation techniques to provide flexibility in development of various detection assays, including but not limited to ELISAs, which may be direct, indirect or sandwich-type ELISAs.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) which are also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability; and (2) an approach based on crystallographic studies of antigen-antibody complexes. As used herein, a CDR may refer to CDRs defined by either approach or by a combination of both approaches.

### PFAS Compounds

The United States Environmental Protection Agency (EPA) curates a master list of PFAS compounds on its internet site currently at comptox.epa.gov. Some of the more common PFAS compounds include perfluorononanoic acid (PFNA), perfluorooctanoic acid (PFOA), perfluorooctane sulfonate (PFOS), perfluorohexane sulfonate (PFHxS), perfluorobutane sulfonate (PFBS), and hexafluoropropylene oxide dimer acid (HFPO-DA, also commonly known as GenX). Notably, each of these compounds includes at least one ionizable functional group which will be protonated or deprotonated, depending on the pH of the solution and the pKa value of each individual ionizable group. For the sake of simplicity, the compound names listed above are intended herein to refer to both the protonated and deprotonated forms as well as salts thereof.

In considering the challenges associated with raising antibodies that bind to small molecules, the present inventors recognized that VHH antibodies are well suited for small molecule analysis. A VHH antibody (also known as a “nanobody”) is the antigen binding fragment of antibodies which consist of only a heavy chain and are naturally produced by camelids and sharks. VHH antibodies are expected to overcome many pitfalls encountered with conventional reagents. In the work so far, VHH antibodies often perform comparably to conventional antibodies for small molecule analysis, are amenable to numerous genetic engineering techniques, and show ease of adaption to other immunodiagnostic platforms for use in environmental monitoring (Bever et al., Anal. Bioanal. Chem., 2016, 408(22): 5985-6002).

In a typical conventional process for isolation of VHH antibodies from camelids which bind to a given compound, the camelids are injected with a solution containing the compound and blood is collected from the camelids, from which mRNA is collected and converted into cDNA by reverse transcriptase PCR. The cDNA is amplified and digested to isolate the VHH genes, for incorporation into plasmids and expressed by a bacteriophage, thereby creating a VHH library. The library is then panned to identify the VHH antibodies of interest (Bever et al., infra). Following isolation of VHH antibodies, testing is conducted to determine binding affinity for binding of the VHH antibodies to the compound of interest.

The target material may comprise PFAS compounds, including, but not limited to six of the most common and potentially dangerous PFAS compounds; PFNA, PFOA, PFOS, PFHxS, PFBS, and GenX.

The antibodies provide the basis for a biosensor for detecting PFAS compounds. In one embodiment, the cartridge may be used in conjunction with an ELISA-based recognition assay where specific binding of an antibody (a binding material) to a given PFAS compound generates an immobilized complex which is detected by a recognition molecule such as a secondary antibody or affinity-based molecule conjugated to an enzyme. The enzyme catalyzes a reaction of a precursor molecule to generate a detectable molecule (a detectable material) which is quantifiable by a sensor using electrochemical, spectrophotometric, colourimetric or fluorometric techniques.

In one example of an ELISA-based biosensor assay, an environmental sample is provided for testing of the presence of a PFAS compounds. An anti-PFAS antibody recognizing a PFAS compound is contacted with the sample and incubated under conditions where the resulting complex is immobilized on a substrate. The sample is washed, and a secondary antibody developed to recognize the anti-PFAS compound antibody is added to the sample. The secondary antibody may include conjugated horseradish peroxidase. A peroxidase substrate such as 3,3′,5,5′-Tetramethylbenzidine (TMB) is added to the sample and is converted by the peroxidase to an electrochemically active species which provides an electrochemical signal correlated with the level of PFNA in the sample.

Examples of enzymes and molecules providing electrochemically active molecules (i.e., an electrochemically detectable material) in a field deployable chemical detector are described in U.S. Pat. Nos. 9,689,046 and 10,415,102.

### BRIEF DESCRIPTION OF THE DRAWINGS

In the Detailed Description section below, one or more embodiments of the present technology are described in relation to the attached figures. These embodiments are intended to provide a better understanding of the invention, how the invention may be put into practice, and to demonstrate some of the advantages of the invention. The drawings are not necessarily to scale, emphasis instead being placed upon illustrating the principles of various embodiments of the invention. Similar reference numerals indicate similar components.

FIG. 1a is a perspective view of an embodiment of the cartridge.

FIG. 1b is a cross-section view of the embodiment of FIG. 1a.

FIG. 1c is a side view of the embodiment of FIG. 1a.

FIG. 1d is a bottom view of the embodiment of FIG. 1a.

FIG. 1e is an exploded view of the embodiment of FIG. 1a.

FIG. 1f is an end view of the embodiment of FIG. 1a interacting with a schematic representation of a base.

FIG. 1g is a schematic representation of the electronics interacting with the sensor of the embodiment of FIG. 1a.

FIG. 1h is a perspective view of a base with three embodiments of the cartridge of FIG. 1a.

FIG. 2 is a graph of the results for a proof-of-concept experiment for a direct ELISA detected calorimetrically.

FIG. 3 is a graph of the results for a proof-of-concept experiment for a direct ELISA detected using voltammetry.

FIG. 4 is a graph of the results for a proof-of-concept experiment for a competitive ELISA detected using voltammetry.

FIG. 5 is a graph of the results for a proof-of-concept experiment for a direct bacteria-based assay detected using voltammetry.

FIG. 6 is a graph of the results for a proof-of-concept experiment for a direct bacteria-based assay detected using voltammetry.

FIG. 7 is a graph of the results for a proof-of-concept experiment for a direct bacteria-based assay detected using voltammetry.

FIG. 8 is a graph of the results for a proof-of-concept experiment showing that dried PAPG gives consistent results with a liquid PAPG control.

FIG. 9 is a graph of the results for a proof-of-concept experiment showing that dried As(III) gives consistent results with a liquid As(III) control.

FIG. 10 is a graph of the results for a proof-of-concept experiment showing that dried selenium (VI) gives consistent results with a liquid selenium (VI) control.

FIG. 11 is a graph of the results for a proof-of-concept experiment for a displacement assay, showing that voltammetric measurements using an embodiment of the present disclosure is similarly sensitive to changes in PFOA concentration as a lab-based optical assay in the range of 0.1-100 ppb.

### DETAILED DESCRIPTION

### Introduction

As described in the background section, The widely used ELISA is a relatively complex multi-stage laboratory procedure typically carried out using a 96-well or 384-well plates.

To reduce the complexity, well strips with a smaller number of wells have been proposed previously. However, these strips are small volume and awkward to handle without spillage or cross-contamination, they require the same amount of manual labour as the full plate, and they do not circumvent the need for light-based analytical equipment for assay readout. Furthermore, the altered plate dimensions raise concerns around the thermal stability of their incubation.

Likewise, microfluidic systems overcome some shortcomings, they are significantly more complex to design and manufacture. They often also rely on very small volumes, which can be challenging to manipulate and in the case of trace analysis like for PFAS compounds, could mean that the small sample does not actually contain the compound of interest or target material (i.e., at the part per trillion level you might take a 0.1 mL sample and not have any of the target molecules).

The present technology provides a field-ready apparatus for performing an assay (e.g., an ELISA) to detect particular target materials that will avoid the manufacturing and usability issues of microfluidics (plus the risk of missing the target analyte when working at ultra-trace levels). The present technology integrates the assay into a sealed cartridge format. This cartridge system allows the user to mix with rotation/shaking (e.g., no pipetting up and down to mix). The cartridge can be sealed closed such that the contents are protected by a shock-proof, leak-proof containment (e.g., the user can drop the cartridge, etc.), with ports to access solution for reagent change steps The cartridge can accommodate greater volumes than typical microfluidic systems, reducing the requirement of fine manipulation on the part of the end user and allowing greater probability of the target analyte being present.

Various aspects of the invention will now be described with reference to the figures. For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that, according to the knowledge and judgment of persons skilled in the art, such alternative features may be substituted in various combinations to arrive at different embodiments of the present invention.

### Cartridge

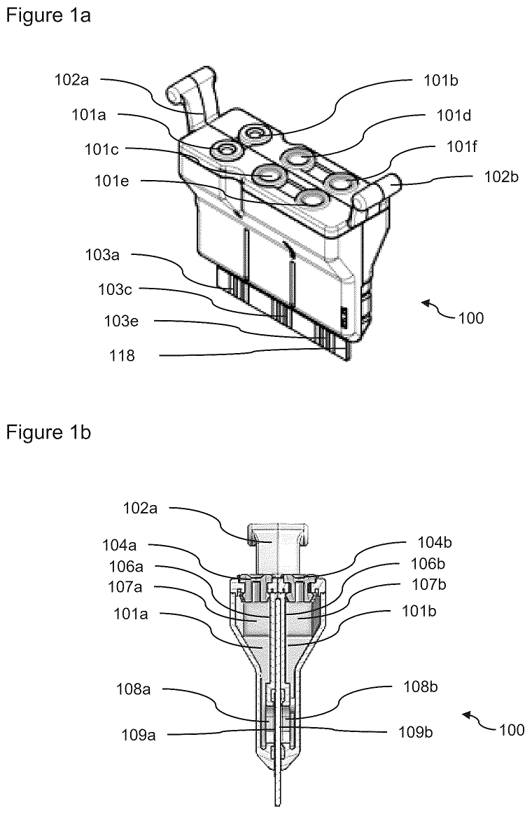

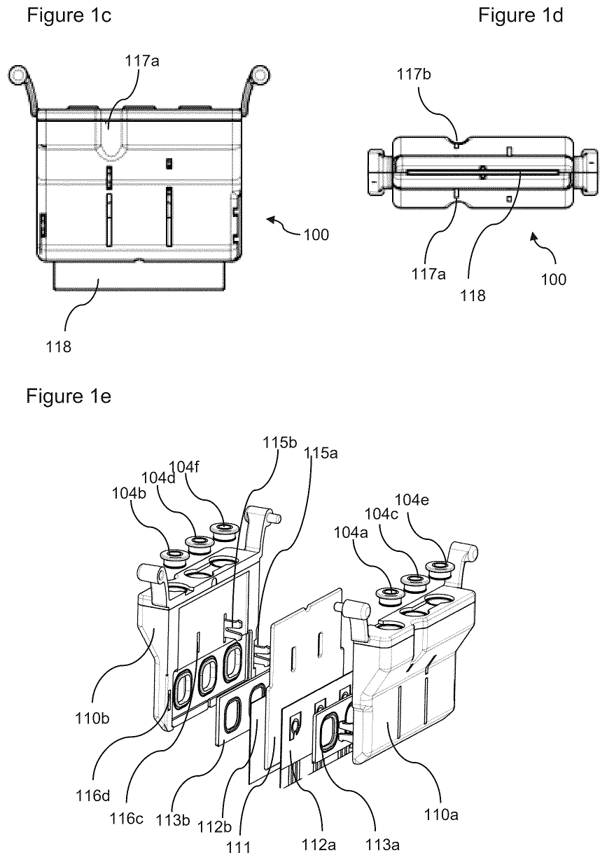

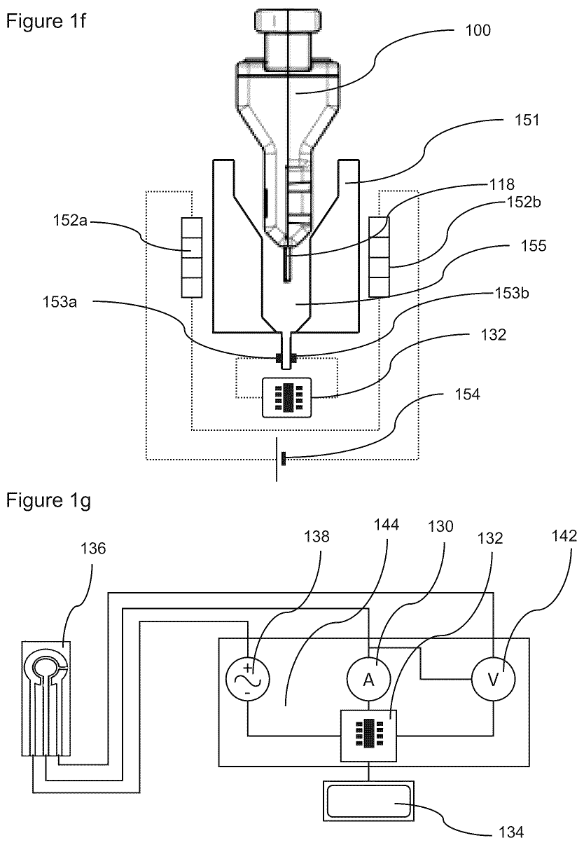

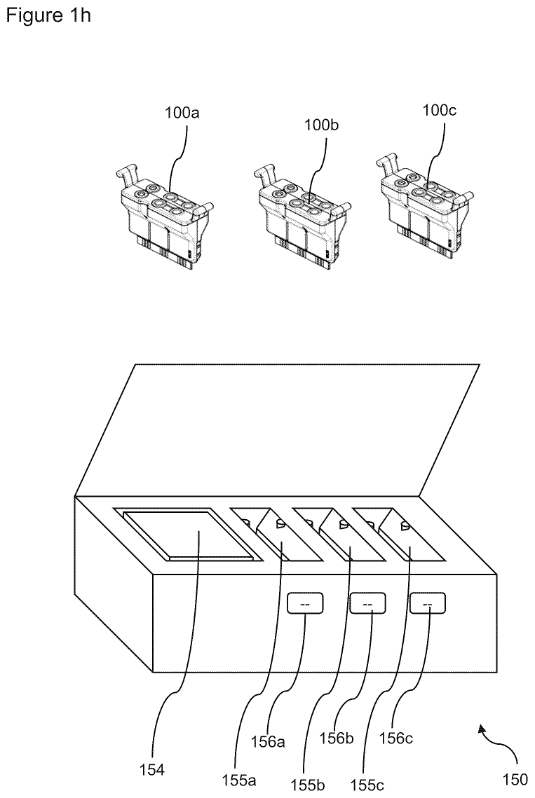

FIGS. 1a-1d are various views of a first embodiment of a cartridge 100, FIG. 1e is an exploded view of the embodiment of FIG. 1a, and FIGS. 1f and 1g are schematic views of the embodiment of FIG. 1a interacting with electronics and a heater housed in a base.

As shown in FIG. 1a, this embodiment comprises six reservoirs 101a-f formed together in a single unitary cartridge, each reservoir being in fluid isolation from each other. The reservoirs are arranged in two rows of three. The cartridge in this case is substantially symmetrical about a vertical mirror plane with respective reservoirs forming pairs on either side of the mirror plane. Handles 102a,b at either end of the cartridge facilitate easy handling in the field. At the bottom of the cartridge, each reservoir has a set of electronics contacts 103a,c for connecting to a corresponding base to allow the base to communicate with and control the sensors (e.g., the sensor electrodes) housed within the cartridge. An elongate reservoir axis is aligned with the rows of reservoirs. In this case, it may be considered to be a horizontal (when the cartridge is in the in-use orientation) axis in the mirror plane.

As shown in FIG. 1b, each reservoir comprises a lid 104a,b at the top (i.e., in the in-use orientation) which is used to seal the inlet into the reservoir. The reservoir comprises a broader processing compartment 107a,b which is located between the inlet and a narrower sensor compartment 108a,b. In this embodiments, the processing compartment comprises a region of binding material 106a,b on an inner surface which interacts to bind the target material. In other embodiments, the binding material may be located elsewhere in the reservoir. In still other embodiments, some assays may not require a binding interaction (e.g., solution-based bacterial assays).

In this embodiment, the sensor compartment may be considered the volume of the reservoir which is within the range of the sensor. In this case, the sensor compartment is defined by: an inner sensor surface with a corresponding sensor 109a, a parallel opposing surface, and an enclosing wall. In this embodiment, the enclosing wall is in the shape of a square with rounded corners (as shown in FIG. 1e).

In this embodiment, the volume of the sensor compartment is around 200 μl (e.g. between 100-300 μl). The distance between the sensor surface and the opposing surface inside the sensor compartment may be between 1-5 mm. The dimensions of the sensor surface along the plane of the sensor surface may be between 0.5-2 cm. To allow free flow throughout the reservoir, any gap between opposing surfaces may be at least 0.5 mm.

The processing compartment in this case is larger and has a volume of around 1.7 ml (e.g., between 1-3 ml). The volume of the processing compartment may be considered to be the volume of the reservoir between the sensor compartment and the inlet, and where the binding material is located. In this case, the processing compartment extends to the bottom of the tapered section.

The inside dimensions of the processing compartment, height, length (i.e., horizontal dimension aligned with the mirror plane) and width (i.e., horizontal dimension transverse to the mirror plane), are each between 0.5-2 cm. In this case, the processing compartment comprises a tapered bottom connecting with the sensor compartment. This allows fluid to flow freely between the sensor and processing compartments (e.g., during filling and/or mixing through shaking). The total volume of the reservoir is around 2 ml (e.g., between 1-3.5 ml).

As shown in FIGS. 1a and 1e, the cartridge is formed from a variety of components. In this case, the cartridge comprises: two main reservoir sections 110a,b, a central backing plate 111, a lid for each reservoir 104a-f, and two electrode sheets 112a,b comprising sensors for each side and electronics connectors. In this embodiment, the electrode sheets are formed from a plastic sheet with electrodes formed by screen printing conductive ink. In other embodiments, PCBs and similar materials (e.g., FR4 CCB) could be used. Gaskets 113a,b are TPU rubber gaskets to ensure a watertight seal. They are over moulded into the cartridge itself and shown as separate in this figure for illustrative purposes.

In this embodiment, the two main reservoir sections each comprise complementary snap-fit connectors 115a,b, 116c,d. As shown in FIG. 1e, two edge snap-fit connectors directly to each other at either end of the cartridge, and two central snap-fit connectors pass through the central backing plate to connect to the other main reservoir section. In this embodiment, each of the two main reservoir sections comprise two male snap-fit connectors 115a,b and two female snap-fit connectors 116a,b. The other components in this case, such as the electronics boards and the two sensor compartment enclosing wall sections are held in place by being in recesses sandwiched between components held together by the snap-fit connectors.

Other embodiments may also have alignment pins that extend from one half, through the electrodes and dividers, and into sockets on the other half. This would allow us to more easily align the sandwiched components, such as the electrodes.

### Sensor

In the embodiment of FIG. 1a, the sensor is a voltammetric sensor. In general, voltammetric assays involve the application of a voltage to a sample fluid containing an electrically active chemical detectable material, and the subsequent detection and evaluation of an electrical current.

FIG. 1f is an example block diagram for a configuration of voltammetric electronics 144 and output interface 134, coupled to voltammetric sensor 136. Voltage source 138 is coupled to voltammetric sensor 136 and to controller 132 which can control voltammetric sensor 136 to apply a voltage to voltammetric sensor 136. When voltammetric sensor 136 is in contact with an electroactive detectable material, for example, a voltage is applied to the cartridge which causes an electric current (i.e., electrical signal) to pass through voltammetric sensor 136 and be detected by current detector 140 while the applied voltage can be detected by voltage detector 142. Current detector 140 and voltage detector 142 are also connected to controller 142. Controller 132 can provide measurements of the detected electrical currents and applied voltage to output interface 134.

The output interface 134 may be connected to controller 132 using a wireless or a wired connection. It will be appreciated that the output interface 134 may or may not form part of the base. E.g., the Output interface may be part of the base or may be part of a remote computer (e.g., a mobile device such as a laptop or cellphone). Output interface 134 may be a display, for example, a liquid crystal display (LCD) or a light emitting diode (LED) display, or be provided in the form a series of indicator LED lights, e.g., green, orange, red to indicate low, medium and high levels of a liquid parameter. Controller 132 includes a processor and/or detection circuitry and may process the detected electrical current and applied voltage to perform a measurement of the target material which may then be shown by the output interface 134.

In detecting or measuring the detectable material, the detected current can be compared to a threshold to ascertain the presence and/or concentration of the target material in the fluid sample. Controller 132 may be further be coupled to a memory device or include a memory element (not shown) in order to store one or more of the measured detected electrical current, the measured detected applied voltage and the measured target material. The memory device may be removably coupled to housing 144, for example, via a Universal Serial Bus (USB) connection. Controller 132 may comprise a transmitter for transmitting data to a remote computer.

### Use of the Cartridge in the Field

The cartridge system of FIG. 1a and associated methods that we have demonstrated can be used with ELISA systems, both competitive and direct ELISAs, with an electrochemical readout, reducing or avoiding the need for the use of 96-well plates. Such a system is much more robust for field deployment, as it can be dropped/shaken without the sample being lost. We have also demonstrated that these cartridges can be filled/emptied with easy-to-use syringes with blunted cannulas (important for field use not to have sharps), which do not require the level of training and dexterity that micropipettes do.

For the embodiment of FIG. 1a, one or more calibration reservoirs are used for in-field calibration curves and one or more test reservoirs for detection of the sample. Most commonly, four chambers are used for calibration curves and two chambers are used for duplicate test determinations of the sample of interest.

For the test determinations a portion of unaltered sample may be injected into the reservoir.

For the calibration chambers there are multiple options on how to generate calibration data. A first option is to add known amounts of the target (a method known as standard addition). A second option is to remove the target (e.g., through oxidation or selective filtration) and then artificially spike in known concentrations of the analyte (allowing “known” concentrations within the background matrix). The second option allows fluids from the same source to be used in both the calibration and test reservoirs. This means that any artefacts from other materials in the sample are present in both the calibration and test reservoirs (e.g., murky water for optical sensors, or electroactive non-target materials for voltammetric sensors, compounds that either inhibit the full normal functioning of the materials used in the reservoir, such as a toxin that slows growth/protein expression for bacteria or something like extra food/sugar that would accelerate growth and protein production).

The processing material interacts with the target material within the test and calibration reservoirs to produce detectable material from the detectable material precursors. In a bacterial assay, the processing material is a bacteria configured to produce the detectable material in response to the presence of the target material. In an ELISA, the processing material typically comprises an enzyme configured to produce the detectable material from the detectable material. In a direct ELISA, the processing material binds onto the target material. In a competitive ELISA, the processing material may compete with the target material in the sample to bind to a binding material. In a direct ELISA, the amount of detectable material is positively correlated with the amount of target material in the sample. In a competitive ELISA, the amount of detectable material is negatively correlated with the amount of target material in the sample. The processing material and/or detectable material precursor may be added to the test and calibration samples by the user or be already present in the reservoir.

The calibration points are used to provide a calibration curve of the response of the sensor as a function of known concentrations of target material. This calibration curve is then used to calculate the concentration of the sample in the duplicated test reservoirs (e.g., through interpolation and/or extrapolation of the calibration data). Using multiple test reservoirs (e.g., in duplicate or triplicate) results in increased accuracy over singlet measurements. In this embodiment, the calibration reservoirs are provided with a known quantity of target material. Information identifying the known quantity (e.g., absolute quantity of target material in reservoir and/or concentration) of target material for each calibration reservoir may be communicated to the base when the cartridge is inserted into the base so that the base may calculate how the response of the sensor varies based on the amount of target material present in the reservoir.

It will be appreciated that this is a tunable process and other numbers of calibration reservoirs and test reservoirs may be used. For example, there may be five calibration reservoirs to one test reservoir, or three calibration reservoirs to three test reservoirs so on.

For the embodiment of FIG. 1a, once the samples are inserted into the reservoirs, the cartridge is inserted into a base as shown in FIG. 1f. The base comprises a heater block 151 (e.g., formed from aluminium or of plastic (e.g., 3D printed plastic)) which has a complementary shape of the outside of the reservoirs. That is, the heating block has a tapering Y-shaped recess 155 for receiving the cartridge such that heat can be transmitted through conduction from the heating block to the outer walls of the processing compartment and to the outer walls of the sensor compartment. The tapering recess comprises an upper upright section configured to about the upright outer wall of the processing section, a lower upright section configured to abut the upright outer wall of the sensor section, and a tapering wall positioned between the upper and lower upright walls and configured to abut the tapering bottom wall of the processing section. This allows the contents of the reservoir to be efficiently heated during any incubation steps required to generate the detectable material from the target material.

In this embodiment, each side of the heating block is heated by a separate heater 152a,b powered by a power source (e.g. battery 154). In other embodiments, a resistive wire may be wrapped around the perimeter of the metal block, providing joule heating from 4 sides with a single heat source. In some embodiments, the thermal conductivity of the block may allow sufficiently consistent heating from a point source. E.g., a point source heating may work best for embodiments in which the heating block is formed from metal (and/or has a heat conductivity of at least 10 W/mK, at least 50 W/mK or at least 100 W/mK).

In this case, although the cartridge has mirror symmetry (e.g., or at least the reservoirs have mirror symmetry), the provision of indents 117a,b (as shown in FIGS. 1c and 1d) means that the cartridge does not have rotational symmetry. Complementary projections in the base recess means that the cartridge can only be inserted into the base in one orientation. In this embodiment, this means that the calibration reservoirs and the test reservoirs are always in the same position within the base.

At the bottom of the cartridge, a cartridge electronics connector 118 is configured to engage with a complementary base electronics connector. This connection allows the base to communicate with each of the individual sensors within each of the respective reservoirs using base electronics contacts 153a,b. The connector 118 is a single dual sided connector. In other embodiments, the side may have a separate connector.

Because the cartridge can only be inserted into the base in one orientation, the electronics 132 of the base can identify that certain reservoirs are manufactured to hold standards, whereas other reservoirs within the cartridge are just the sample itself. This enables the generation of the calibration curve within the target matrix itself as opposed to periodic calibration cartridges that do not account for matrix interferences.

FIG. 1h is a perspective view of a portable assembly which can be used in the field comprising a base 150 and multiple cartridges 100a-c. The base comprises multiple recesses for receiving the cartridges 155a-c. The base also comprises a power source (e.g., battery 154) for powering the electronics and the heaters and/or coolers used to control the temperature in the recesses. Each recess is associated with an output interface display 156a-c for displaying the results for cartridges connected to each of the three recesses. In other embodiments, the base may comprise a single display for showing all the results. Other embodiments may transmit data electronically (e.g., wirelessly via WiFi, or to a data storage device such as a USB). The unit also comprises a cover for closing over the top of the cartridges when connected to the recesses. This helps make the assembly portable, and also helps control the temperature within the cartridge while the cartridges are being incubated. The cover may be insulated to reduce heat loss to the environment, e.g., from the block heaters. In other embodiments, an insulated lid may be provided to cover the recesses, and a separate overall lid (which may or may not be insulated) to enclose the base (e.g. including the battery and/or user interface).

In some cases, there may be a pretreatment of the sample before it is placed in the reservoir. For example, a particular processing material may be sensitive to a particular form of a material. E.g., the user may wish to detect the total amount of arsenic, whereas the processing material (e.g., a bacteria) may be sensitive only to As(III). In this case, a pretreatment of a test sample may be used to convert As(V) present in the sample to the target material As(III). In this example, the pretreatment may comprise adding a reducing agents and/or acid to the original sample to convert As(V) to As(III). A neutralizing agent may be subsequently added before the treated sample is added to the cartridge so that cartridge is not acidified and/or so as not to overwhelm the buffer.

For a calibration determination, a calibration pretreatment may be performed on the original sample to alter any target material present in the original sample so that it not detected by the processing material. For example, where the processing material is sensitive only to As(III), the calibration pretreatment may comprise adding a oxidant (e.g. MnO₂) to convert all As(III) into As(V), thereby removing the As(III) from the original sample and forming a calibration sample. The calibration sample is then injected injected into the calibration chambers in the cartridge, which have known quantities of arsenic present in them.

### Experimental (ELISA)

A number of ELISA tests are described below to demonstrate that they could be used in conjunction with the cartridges described above. The first test includes a colorimetric direct ELISA of a capture protein that is detected with a horseradish peroxidase (HRP) conjugated antibody and developed with the substrate TMB. The second one uses an electrochemical readout of current change, using an alkaline phosphatase conjugate antibody with the substrate p-aminophenol phosphate.

Colorimetric Direct ELISA (dELISA)

In a direct ELISA, the target material, or antigen, is immobilized directly onto the surface of the cartridge within the processing compartment, and then complexed with an enzyme-labeled primary antibody specific for the antigen. Once the enzyme-labeled primary antibody binds to the antigen, the conjugated primary antibody catalyzes a reaction with its respective substrate resulting in a product (i.e., a detectable material) that, for example, may be measured by an optical sensor such as a spectrophotometer or absorbance sensor. Direct ELISAs are suitable for qualitative and quantitative target material detection. It will be appreciated that, for coloured detectable materials, a transparent window into the cartridge will allow the user to visually monitor the production of the coloured detectable material. The transparent window may be positioned on a top surface of the cartridge which is visible when the cartridge is inserted into the base.

The proof-of-concept method carried was carried out using the following steps:
1. Cartridge plastic was treated with 0, 1, or 100 nM of a HA-tagged VHH nanobody suspended in 100 mM, pH 8 sodium carbonate buffer solution for 2 hours in a humidified chamber. Following rinse of excess capture molecule three times with 0.05% Tween-20 in PBS, the remaining plastic surface was blocked with 2% skim milk in PBS for one hour to prevent nonspecific binding. In an actual working embodiment, this capture coating and block would be performed by the manufacturing facility and stably stored until ready for use in the field.
2. Following rinse of excess blocking solution three times with 0.05% Tween-20 in phosphate-buffered saline (PBS), the cartridge cell was treated with an HRP conjugated anti-HA antibody at a 1:20,000 dilution in PBS for one hour.
3. The cartridges were then rinsed four times with 0.05% Tween™-20 in PBS and then filled with 1×TMB developing solution that turns blue upon HRP exposure. The system was allowed to develop for 5 minutes before the addition of a 1 N HCl stop solution.
   Electrochemical dELISA and Competitive ELISA (cELISA)

A second series of two proof-of-concept ELISA experiments was carried out to use electrochemical sensing rather than optical sensing.

This series of experiments used both direct and competitive ELISAs.

In competitive ELISA, also referred to as inhibition ELISA, the concentration of the target antigen (or target material) is determined by detection of signal interference. The target antigen within the sample competes with a reference antigen for binding to a specific amount of labeled antibody. The reference antigen is pre-coated on a multi-well plate and sample is pre-incubated with labeled antibody and added to the wells. Depending on the amount of antigen target material in the sample, more or less free antibodies will be available to bind the reference antigen detectable material. This means the more antigen target material there is in the sample, the less reference antigen detectable material will be detected and the weaker the signal. This is an example where the presence of the target material controls the production of the detectable material by decreasing the production of detectable material.

The proof-of-concept method carried was carried out using the following steps:
1. The cartridge plastic was treated with 0, 0.1 or 1 μg/mL of BSA as the capture layer in the same manner as with the colorimetric dELISA. For the cELISA, only 1 μg/mL of BSA was used.
2. Following rinse of the blocking buffer three times with 0.05% Tween-20 in PBS, the cartridge was treated with a sheep anti-BSA IgG antibody in the dELISA at a 1:20,000 dilution in PBS. For the competitive ELISA, it was simultaneously treated with the sheep anti-BSA IgG antibody at a 1:20,000 dilution and free BSA that will compete with the BSA capture layer for antibody binding (testing concentrations of 0, 0.001, and 10 μg/mL of free BSA). All incubations were for one hour.
3. Following incubation and rinsing three times with 0.05% Tween-20 in PBS, all samples were treated with an AP conjugated anti-sheep IgG antibody that recognizes the sheep anti-BSA IgG at a 1:10,000 dilution in PBS for one hour.
4. After four final rinses with 0.05% tween-20 in PBS, all cartridges were developed for 1 hour with 1 mg/mL p-aminophenol phosphate developing solution in 1×DEA prior to the addition of a 1 N HCl stop solution and scanning via cyclic voltammetry and measuring the corresponding peak height due to the production of p-aminophneol.

### Results

The inventors were able to successfully create a capture layer of binding material on the walls of a cartridge in all three ELISA experiments performed. This indicates that the wall material is compatible with the use of ELISAs. The inventors found that that the conjugate antibody (a detectable material precursor) was selectively captured in a targeted region where the antibody was deposited. This was evident through the colourimetric development of TMB in the 100 nM test, because visual inspection showed that the colour appeared only in the marked circle where the antibody was deposited (with a development time of 1 minute 45 seconds). That is, the substrate colour conversion occurred directly over the region of the droplet deposition and not in other regions of the cartridge. Additionally, the control that lacked capture protein did not show appreciable substrate development.

Regarding the electrochemical series of tests, using a direct and competitive ELISA using the conjugate antibody AP and substrate p-aminophenol phosphate, the inventors observed see changes in current that indicated successful binding of the target material to the surface in the direct ELISA, and that the signal could be suppressed with the competitive ELISA.

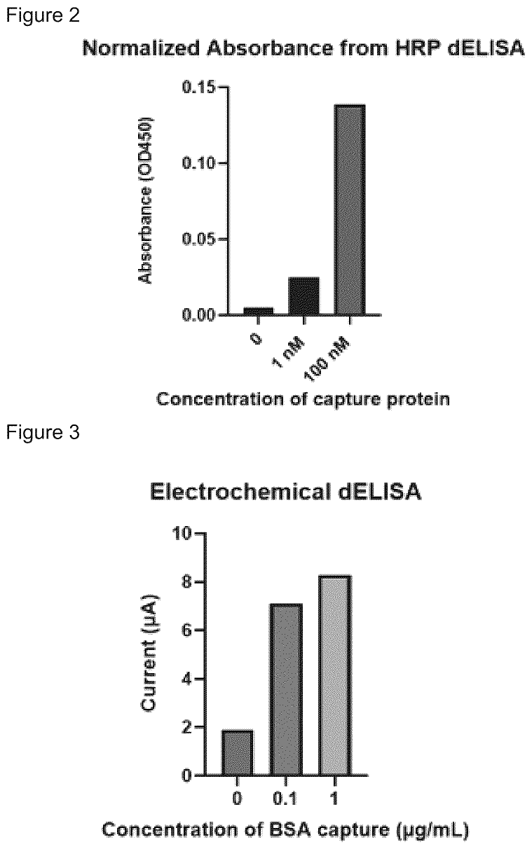

FIG. 2 is a graph of the OD450 values for three samples removed from the cartridges in the HPR dELISA experiment as a function of the concentration of capture protein. The OD value is measure of how much of the yellow colour has been produced. The concentration of colour produced is proportional to the amount of detectable material that was present in the sample. Results are expressed as Optical Density (OD₄₅₀) measurements using a microplate reader with a 450 nm filter. The values were measured in a tube container, and the values plotted are those after the OD₄₅₀ value of an empty tube container was subtracted. As shown in FIG. 2, the optical density varies as a function of capture protein.

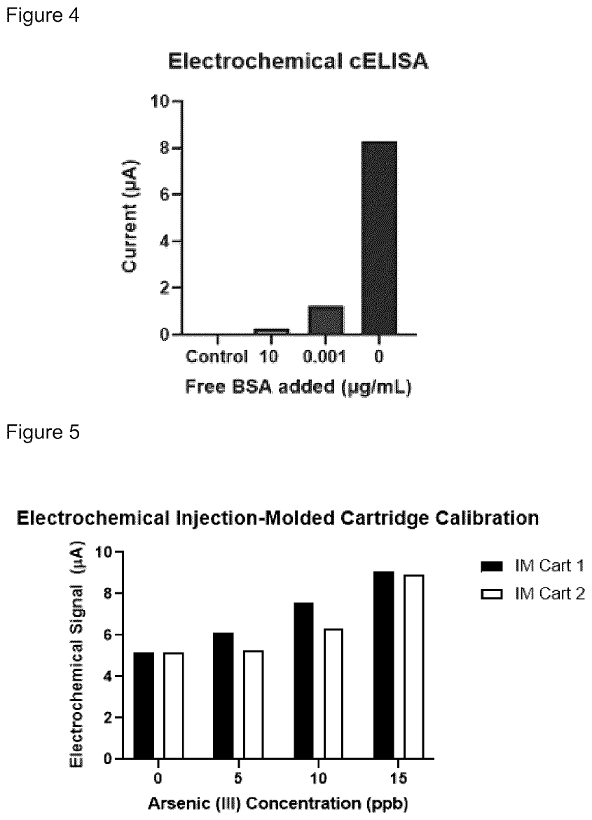

FIGS. 3 and 4 are graphs of electrochemical detection corresponding to respectively the direct ELISA and competitive ELISA experiments performed with BSA as a capture layer. Detection was performed with p-aminophenol phosphate as the detectable material, as a substrate of AP. Current was measured after 1 hour via cyclic voltammetry and the current values were calculated via conventional means.

It will be appreciated that multiple alternative electrochemical techniques may be used, other than cyclic voltammetery. In this example we used standard tools that were provided with the potentiostat from the manufacturer (DropSens™). These tools are used to calculate the oxidation peak current from a cyclic voltammogram and take into account the appropriate baselines.

For FIG. 3, direct ELISA was performed with two concentrations of BSA as a capture layer. The cartridges were subsequently incubated with anti-BSA and an AP conjugate enzyme that recognizes anti-BSA.

For FIG. 4, a competitive ELISA was performed with 1 μg/mL BSA as a capture layer, except for the control (carbonate buffer only). The capture layer was challenged with 0, 0.001 and 10 μg/mL of BSA. Because FIG. 4 represents a competitive ELISA, the x-axis is plotted in order of decreasing free BSA added.

As shown in FIGS. 3 and 4, the current response varies as a function of BSA concentrations.

Importantly, our demonstrations have performed the full ELISA protocol within the cartridge. It is common in the field of electrochemistry to “protect” the electrode by avoiding any solution that could coat the surface of the electrode until it is absolutely necessary. If the surface of an electrode is sufficiently coated/blocked, it may be unable to accurately measure the concentration of an analyte in the solution that it is touching. This is known as “fouling”. With our cartridges, we were able to block the surface without removing the electrode's ability to measure what was present in the solution.

### Arsenic Sensing Bacteria

The cartridge may also be used to detect a target material using other assays, other than ELISA.

In this experiment, the inventors used an arsenic-sensing bacterial strain in order to electrochemically detect the presence of arsenic in an aqueous sample.

To test the feasibility of detecting arsenic electrochemically, the following steps were carried out:
1. A culture of a previously described arsenic-responsive bacterial strain (see Sanchez et al., “The Integration of Whole-Cell Biosensors for the Field-Ready Electrochemical Detection of Arsenic”, Journal of The Electrochemical Society, (2021) 168, 067508, hereby incorporated by reference in its entirety) was grown overnight to stationary phase in LB and then diluted 1:100 and grown as a subculture for 3 hours in fresh LB prior to testing.
2. 200 μl of the biosensor subculture was thoroughly mixed with 900 μl of water containing between 0-20 ppb of As(III) and solid p-aminophenyl-beta-D-galactopyranoside (PAPG, final concentration 0.88 mM) within the cartridge and the mixture was incubated at 37° C. for 2 h.
3. The response to arsenic was measured using a potentiostat and screen-printed electrodes via cyclic voltammetry and observing the corresponding oxidation peak height due to the production of p-aminophneol.

FIG. 5 shows the electrochemical response signal (e.g., current response) for the two cartridges. As shown in FIG. 5, both cartridges show a response which varies as a function of arsenic concentration. However, there is some variability between the two cartridges.

In an actual working embodiment, where multiple reservoirs are used to determine the concentration of a target material in multiple samples, the responses of the multiple cartridges may be averaged (e.g., using a mean average) to determine a more accurate value for the concentration of the target material. The variability between the multiple cartridges may also be used to calculate an error figure for the determined concentration value of the target material.

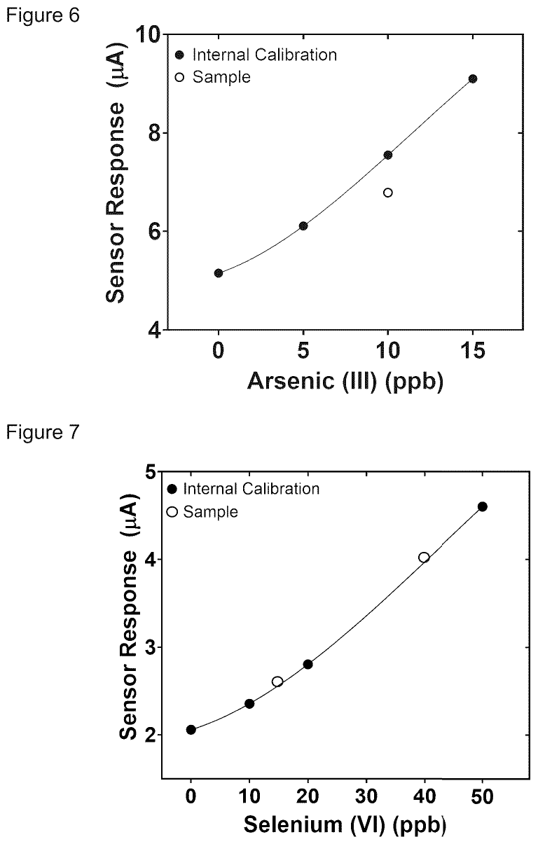

FIG. 6 shows a typical calibration curve which was generated using the present technology for arsenic in the 0-15 ppb range, and how the results for one sample of known concentration is consistent with the calibration curve.

In this case, 160 μL of 5 mM PAPG was diluted in a phosphate buffer/NSF solution (0.4M, pH 7) and dried down in a cartridge overnight at room temperature.

The sensor detects As(III), so water must first be pre-treated to convert As(V) to As(III). 25 mL of 25 ppb arsenic (V) in Milli-Q water was added to a mixer bottle. 962.5 μL of a HCl (6 M) and CuCl₂ (5 mM) solution and 56.3 μl of 0.5 M sodium thiosulfate was added to the mixture, taking the solution to pH 1.07. The solution was then mixed for 20 minutes. The solution was neutralized with 50 mg of PB powder and titrated with 6 M NaOH. The mixture was diluted 2-fold with NSF water, before 3 mL was passed through a 0.45 μm filter. 1 mL of the filtered solution was added to two sample channels. 5 mL of the 2-fold diluted solution with NSF was passed through 80 mg of MnO₂ before being filtered through a 0.45 μm filter.

The MnO₂ treated water oxidizes the As(III) to As(V), so the water can be used in the calibration curve. This water is added along with an As(III) spike to final concentrations of 0, 5, 10 and 15 ppb.

1 mL of each concentration were added to appropriate singlet cartridge channels. The cartridge was inverted for 30 seconds to dissolve the dried PAPG solution. 7 mL of cryoprotectant was added to a 12 day old lyophilized E. coli arsenic sensor cell vial and inverted for 30 seconds. 200 μL of the rehydrated cells were added to each channel in the cartridge and the cartridge was inverted for a further 30 seconds. The cartridge was incubated for 2 hours at 37° C. The cartridge was then measured electrochemically on a potentiostat.

The sample result shows that very accurate results can be obtained for arsenic (III) in the field using the present cartridge system.

### Additional Experiments

### Selenium

FIG. 7 shows a calibration curve which was generated using the present technology for selenium in the 0-50 ppb range, and how the results for two samples of known concentrations are consistent with the calibration curve.

To generate the calibration curves, 160 μL of 5 mM PAPG (p-aminophenyl-beta-D-galactopyranoside) diluted in a phosphate buffer (0.4 M)/NSF solution (pH 7) was dried down in the polycarbonate injection-molded cartridge overnight at room temperature.

Calibration selenium (VI) dilutions were made in Milli-Q water (purified using a Millipore™ Milli-Q™ lab water system) to final concentrations of 0, 10, 20, and 50 ppb and sample selenium (VI) dilutions were made up in Milli-Q water to final concentrations of 14.7 and 40 ppb. 1 mL of each concentration were added to appropriate cartridge channels. The cartridge was inverted for 30 seconds to dissolve the dried PAPG solution. 7 mL of cryoprotectant was added to a 45 day old lyophilized E. coli selenium sensor cell vial and inverted for 30 seconds. 200 μL of the rehydrated cells were added to each channel in the cartridge and the cartridge was inverted for a further 30 seconds. The cartridge was inserted into a FRED-detector and was incubated for 3 hours at 37° C. The cartridge was then measured electrochemically on a potentiostat.

The sample result shows that reasonably accurate results can be obtained in the field for selenium (VI) using the present cartridge system.

Dried PAPG Week 4-time course with Arsenite 10 ppb

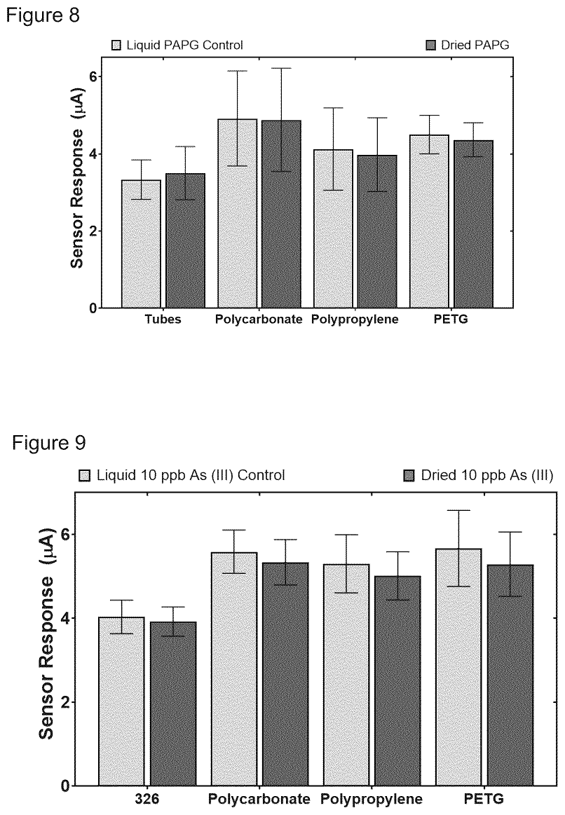

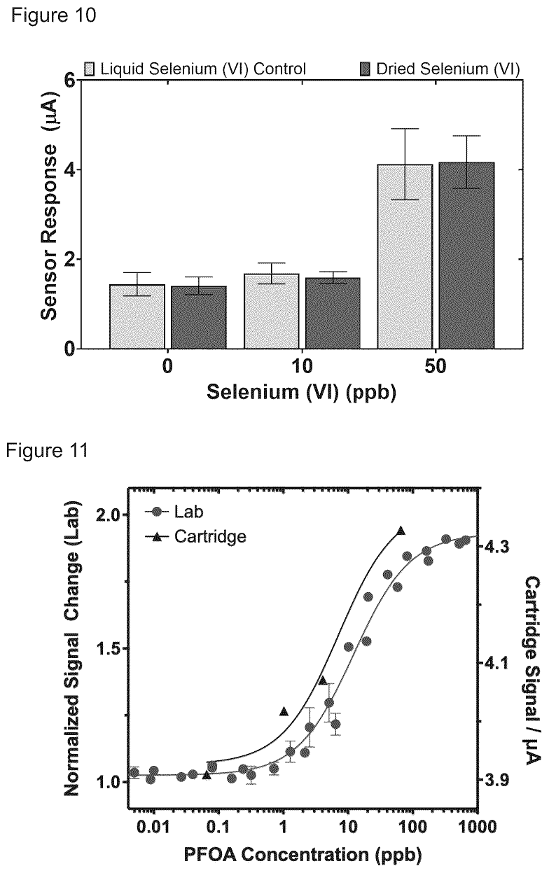

FIG. 8 shows sensor responses for 10 ppb arsenite, comparing a liquid PAPG control and dried PAPG which has been stored for four weeks, and four different cartridge materials (conventional tubes, polycarbonate, polypropylene and polyethylene terephthalate glycol (PETG)).

160 μL of 5 mM PAPG diluted in a phosphate buffer (0.4 M)/NSF solution (pH 7) was dried down in polycarbonate injection-molded cartridge ‘dried PAPG’ channels overnight at room temperature. The cartridges were then stored at 4° C. for 4 weeks. 160 μL of 5 mM PAPG diluted in a phosphate buffer (0.4 M)/NSF solution (pH 7) was added to the ‘liquid PAPG control’ channels.

Arsenic (III) dilutions were made in Milli-Q water to final concentrations of 10 ppb. 1 mL of 10 ppb As(III) was added to the ‘dried PAPG’ cartridge channels and 840 μL was added to the ‘liquid PAPG control’ channels. The cartridge was inverted for 30 seconds to dissolve the dried PAPG solution. 200 μL of subcultured E. coli arsenic sensor cells were added to each channel in the cartridge and the cartridge was inverted for a further 30 seconds. The cartridge was incubated for 2 hours at 37° C. The cartridge was then measured electrochemically on a potentiostat.

This shows that dried PAPG gives results which are consistent with the liquid PAPG control. This indicates that cartridges with dried PAPG may be stored for a considerable period of time, and still give consistent results.

### As(III) 10 ppb Storage

FIG. 8 shows sensor responses for 10 ppb As(III), comparing a liquid 10 ppb As (III) control and dried As(III) which has been stored for four weeks, and four different cartridge materials (conventional tubes (326—formed from a drop-casted urethane material), polycarbonate, polypropylene and polyethylene terephthalate glycol (PETG)).

Filter paper and a As(III)/cryoprotectant mix was added to the ‘Dried 10 ppb As(III)’ channels for a final concentration of 10 ppb. The As(III) standard was then lyophilized and the cartridges were then stored at 4° C. for 4 weeks. 160 μL of 5 mM PAPG diluted in a phosphate buffer (0.4 M)/NSF solution (pH 7) was added to all of the channels. Arsenic (III) dilutions were made in Milli-Q water to final concentrations of 10 ppb. 1 mL of Milli-Q water was added to the ‘dried 10 ppb As(III)’ cartridge channels and 840 μL of the As(III) dilution was added to the ‘liquid 10 ppb As(III) control’ channels. The cartridge was inverted for 30 seconds to dissolve the dried PAPG solution. 200 μL of subcultured E. coli arsenic sensor cells were added to each channel in the cartridge and the cartridge was inverted for a further 30 seconds. The cartridge was incubated for 2 hours at 37° C. The cartridge was then measured electrochemically on a potentiostat.

This shows that dried As(III) gives results which are consistent with the liquid As(III) control. This indicates that cartridges with dried As(III) may be stored for a considerable period of time, and still give consistent results.

### Dried Selenate in PAPG, 1 Day Storage

FIG. 8 shows sensor responses for selenium, comparing a liquid selenium (VI) control and dried selenium (VI) which has been stored for four weeks, at three different concentrations (0, 10 and 50 ppb).

160 μL of 5 mM PAPG diluted in a phosphate buffer (0.4 M)/NSF solution (pH 7) was dried down in duplicate polycarbonate injection-molded cartridge channels. Milli-Q or a concentrated stock of Se(VI) made in Milli-Q was added to the ‘Dried Selenium (VI)’ channels, along with the PAPG, for final concentrations of 0, 10, and 50 ppb. The cartridges were dried overnight at room temperature. Se(VI) dilutions were made in Milli-Q water to final concentrations of 10 and 50 ppb. 1 mL of Milli-Q water was added to the ‘dried Selenium (VI)” cartridge channels and 1 mL of the Se(VI) dilutions were added to the ‘liquid Selenium (VI) control’ channels. The cartridges were inverted for 30 seconds to dissolve the dried PAPG solution. 7 mL of cryoprotectant was added to a 17 day old lyophilized E. coli selenium sensor cell vial and inverted for 30 seconds. 200 μL of the rehydrated E. coli selenium sensor cells were added to each channel in the cartridge and the cartridge was inverted for a further 30 seconds. The cartridge was incubated for 3 hours at 37° C. The cartridge was then measured electrochemically on a potentiostat.

This shows that dried selenium (VI) gives results which are consistent with the liquid selenium (VI) control. This indicates that cartridges with dried selenium (VI) may be stored for a considerable period of time, and still give consistent results.

### Indicator Displacement Assay (IDA)

FIG. 11 shows a comparison between two indicator displacement assays: one performed in a lab setting using spectrophotometery, and an assay performed in a cartridge using voltammetry. Both assays show the response to different concentrations of PFOA.

For the lab assay (shown in circles), a 20,000 ppm of PFOA stock was prepared in 2 mL of HPLC grade methanol in a glass vial. The stock solution was then used to prepare serial dilution series.

After mixing and vortexing, each serial dilution was mixed 10 times with a pipette and 20 μL was pipetted out and added to PCR tubes containing 80 μL of the indicator displacement assay (IDA) mix containing 0.10 mM of p-β-cyclodextrin combined with 0.008 mM indicator die (8-anilinonaphthalene-1-sulfonic acid (ANS)) in water.

The solution was then mixed using a vortex and incubated at room temperature for 2 hours in a box to prevent photobleaching. 90 μL of each solution was then transferred into a black plate, and measured using the spectrophotometer (excitation of 375 and emission of 477 nm).

For the cartridge assay (shown in triangles), a 20,000 ppm of PFOA stock was prepared in 2 mL of HPLC grade methanol in a glass vial. The stock solution was then used to prepare different dilution: 100, 10, 1, 0.1 and 0.01 ppm PFOA.

After mixing and vortexing, 20 μL was pipetted out and added to PCR tubes containing 80 μL of the IDA mix containing 2.0 mM of p-β-cyclodextrin combined with 0.1 mM of ferrocenemethanol (FcMeOH). The solution was then mixed using a vortex and incubated at room temperature for 2 hours. 100 μL of the solution was then transferred in the cartridge and measured using voltammetry using a base as described above. The signal is acquired with square wave voltammetry; the cartridge signal intensity is measure by determining the peak height.

These graphs show that the cartridge voltammetric signal provides a measurable dependence on PFOA concentration comparable to that of the lab determined spectrophotometry signal across at least the range of between 0.1-100 ppb.

### Other Design Features

In other embodiments, each reservoir may comprise an outlet to simplify removal of solutions from the cartridge. The outlet may comprise a Luer-lok™ port. The inlet may function as an outlet. The outlet may be a dedicated outlet, separate from the inlet, to fluid flow through the cartridge (e.g., injected into the inlet and removed at the outlet). This may improve the ease-of-use and reduce user contact with the sample (for safety and assurance of assay performance).

Multiple other materials could be utilized, based on ease of manufacture, material properties and affinity to absorbing binding material. As well as polycarbonate, it is known that polystyrene can be injection molded and has good affinity to adsorbing proteins on its surface for ELISA applications. There are also a range of other materials, such as polyamides and poly-ones (polysulfones, polyketones, etc.) that would be amenable to injection molding and may provide acceptable adsorption levels. Materials made of polyolefins (PP/PE) may also be used, as they are common materials in injection molding systems. However, they may not be as good an affinity to absorbing proteins specifically for ELISA applications, although their utility in other applications (e.g., bacterial assays) could be advantageous.

The base may comprise a camera to detect colours or colour patterns of cartridges inserted into the recesses to identify reservoirs. The camera would be connected to the electronics unit. Colours may be used to distinguish reservoirs used for calibration and reservoirs used for testing an actual sample. Colours may be used to distinguish different target materials (e.g., yellow for arsenic, orange for copper, black for PFAS).

Each cartridge and/or reservoir may carry a code, such as an RFID tags or QR code. The base may have a corresponding reader for reading these codes. The codes may be used to identify the type of assay, the target material and/or whether the reservoir is used for calibration or testing.

The base may comprise a location sensor (e.g., GPS) for determining the location of the base. The base may comprise a clock for keeping time. The base may be configured to associate the location and time of the test to each determined result.

The binding material may be mounted on beads within the processing compartment. The beads may be magnetic to allow them to be controlled using a magnetic field.

The detectable material may be a soluble redox and/or chromogenic species.

The kit may comprise syringes for transferring fluids. There may be different syringes and/or vessels for test determinations and calibration determinations. The syringes may contain pretreatment materials (e.g., oxidising or reducing agents). The syringes may have filters for removing solids from the liquids. The solids may be materials within the sample (e.g., grit from the environment from where the sample was taken) and/or materials used as part of the processing of the sample (e.g., oxidising or reducing agents).

### ELISA Optimization

Adjustments to both the process and material compositions can be pursued to further optimize the ELISAs, including when alternate targets (e.g., non-proteinaceous analytes) are of interest. To improve the process, incubation times at different steps may be adjusted to determine the minimum time needed for a desired sensitivity. As mentioned in the cartridge design adjustments, different solution exchange methods can be explored to improve the ease-of-use of blocking and washing, and to enable sampling in more hostile solution environments. Other embodiments may have a binding material in a different location and with a different surface area within the cartridge. Adjusting the environmental conditions may also allow for different materials to be used. For example, at normal temperatures and pressures certain levels of solvent may destroy particular protein structures. Changing the conditions may allow these levels of solvent to be used.

Materials used within the present invention (e.g., processing material, binding material, detectable material precursor) may be positioned on or adjacent to the sensor (e.g., voltammetric sensor) to reduce diffusion distance between the produced detectable material and the sensor.

Another option is to use an alternative matrix like polystyrene beads functionalized with the capture molecule within the processing compartment (e.g., rather than the processing compartment wall itself).

The size and shape of these beads may allow for simple assembly and for the protection of the electrode. For example, 3 mm beads would not be able to fit into the electrode sensing compartment, allowing spatial separation. In addition to this, beads would allow for a greater surface area vs using the cartridge walls. In some ELISA setups, surface area is proportional to sensitivity. Finally, having beads allows for more of the solution to be “touching” what is, in effect, the recognition surface. This could reduce assay time, as otherwise we may be waiting for diffusion from the middle of the cartridge to the walls of the surface, where the diffusion coefficient of most of these molecules will be low (e.g., in the range of 10⁻⁵-10⁻⁶ cm²/s).

Other materials may be used in the process such as drying the capture protein and block in a sucrose matrix, requiring only sample addition to hydrate the assay and start the incubation steps.

The buffer compositions may be adjusted to better accommodate being part of a field kit, avoiding reagents needing refrigeration or having limited shelf-life, and to ensure maximum compatibility with the materials of the electrode and other cartridge components.

Although the present invention has been described and illustrated with respect to preferred embodiments and preferred uses thereof, it is not to be so limited since modifications and changes can be made therein which are within the full, intended scope of the invention as understood by those skilled in the art.

1. A kit comprising:
a processing material;
a detectable material precursor; and
a sample analysis cartridge for the detection of a target material in a fluid sample, the sample analysis cartridge comprising:
multiple sealable reservoirs, each reservoir having:
an inlet for receiving a fluid sample;
a processing compartment in fluid communication with the inlet; and
a sensor compartment being in fluid communication with the processing compartment and being accessible by a sensor, the sensor being configured to provide an output in response to the presence of a detectable material, the detectable material being producible by the processing material from the detectable material precursor,
wherein the kit is configured such that the quantity of detectable material produced by the processing material is correlated to the quantity of the target material in the fluid sample.

2. The kit according to claim 1, wherein the sensor comprises an optical sensor.

3. The kit according to claim 1, wherein a binding material is immobilized on a surface of the reservoir, the binding material being configured to selectively bind to the target material.

4. The kit according to claim 1, wherein the processing material is configured to bind to the target material, and wherein a binding material comprising target material is immobilized on a surface inside the reservoir, such that, when the processing material and sample fluid are added to the reservoir, the processing material binds with the unbound target material in the sample fluid and with the bound target material immobilized on the surface inside the reservoir.

5. The kit according to claim 3, wherein the binding material comprises an antibody or antibody fragment suitable for use in an ELISA.

6. The kit according to claim 3, wherein the binding material comprises at least one of: an IgG antibody; and a VHH antibody.

7. The kit according to claim 1, wherein the processing material comprises bacteria.

8. The kit according to claim 1, wherein the processing material is a cyclodextrin, and the detectable material precursor is a guest molecule bound to the cyclodextrin in a complex, the guest molecule being displaceable by the target material to form an unbound guest molecule detectable material.

9. The kit according to claim 1, wherein the sensor compartment comprises a planar inner surface on which the sensor is mounted, and a parallel opposing surface.

10. The kit according to claim 1, wherein the sample analysis cartridge is rotationally asymmetric.

11. The kit according to claim 1, wherein the reservoirs have mirror symmetry with at least one pair of reservoirs arranged on either side of a mirror plane.

12. The kit according to claim 1, wherein the reservoirs have a common central barrier, the barrier comprising the sensor for each of the reservoirs.

13. The kit according to claim 1, wherein the sample analysis cartridge has a tapered external shape wherein the width of the cartridge around the sensor compartment is narrower than the width of the sample analysis cartridge around the processing compartment.

14. A method of detecting a target material in a fluid sample using a processing material, a detectable material precursor, and a sample cartridge, the sample analysis cartridge comprising:
multiple reservoirs, each reservoir comprising:
an inlet for receiving a fluid sample;
a processing compartment in fluid communication with the inlet; and
a sensor compartment being in fluid communication with the processing compartment and being accessible by a sensor, the sensor being configured to provide an output in response to the presence of a detectable material, the detectable material being producible by the processing material from the detectable material precursor,
the method comprising:
injecting the fluid sample into the reservoir;
enabling production, by the processing material, of the detectable material from the detectable material precursor within the reservoir based on the presence of the target material; and
detecting the response of the sensor to the detectable material.

15. The method according to claim 14, wherein the method comprises:
injecting the fluid sample into the reservoir, the reservoir comprising a binding material configured to selectively bind the target material within the injected sample to the surface within the cartridge;
flushing the cartridge;
adding the processing material and the detectable material precursor;
allowing the detectable material precursor fluid to interact with the intermediate material to produce the detectable material;
detecting the response of the sensor to the detectable material.

16. The method according to claim 14, wherein the method comprises:
simultaneously determining the quantity of target material within multiple reservoirs within a cartridge, wherein at least two of the determinations are calibration determinations, and at least one determination is a test determination,
wherein each calibration provides an association between a known quantity of target material and a sensor response; and
wherein the sensor response of the at least one test determination is interpolated between the sensor responses of the at least two calibration determinations to provide a concentration of the target material.

17. The method according to claim 16, wherein the method comprises removing the target material from a sample fluid to form a calibration fluid, wherein the calibration fluid is used in the calibration determination, and the unaltered sample fluid is used in the test determination.

18. The method according to claim 17, wherein removing the target material from the sample fluid to form the calibration fluid comprises one or more of: filtration, reacting the target material, changing the oxidation state of the target material, changing the physical state of the target material, immobilizing the target material, binding the target material and chelating the target material.

19. A sample analysis cartridge for the detection of a target material in a fluid sample, the cartridge comprising:
multiple reservoirs, each reservoir comprising:
an inlet for receiving a fluid sample;
a processing compartment in fluid communication with the inlet; and
a sensor compartment being in fluid communication with the processing compartment and being accessible by a sensor, the sensor being configured to provide an output in response to the presence of a detectable material, the detectable material being producible by a processing material from a detectable material precursor based on the presence of a target material.

20. The sample analysis cartridge according to claim 17, wherein the cartridge comprises indents positioned between two adjacent reservoirs, the indents being positioned away from the center along a reservoir axis.
